(19) United States
(12) Patent Application Publication
(10) Pub. No.: US 2003/0187457 A1
(43) Pub. Date: Oct. 2, 2003

(54) Apparatus and method for removing an object from a body

(76) Inventor: John A. Weber, Highland Park, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/114,889

(22) Filed: Apr. 2, 2002

(51) Int. Cl. A61B 17/22

(52) U.S. Cl. 606/110

(57) ABSTRACT

An apparatus and a method for removing an object from a body are disclosed. The apparatus may include a sheath member, a snare, a forcep, and a biasing member. The sheath member may include a first channel and a second channel. The snare may be slidably disposed in the first channel of the sheath member. The snare includes a snare loop to encircle the object. The forcep may be slidably disposed in the second channel of the sheath member. The forcep may be oriented to a threaded position relative to the snare loop to engage the object. The biasing member may be operatively associated with the forcep to adjust the forcep between first and second positions for grasping the object.

### FIELD OF THE DISCLOSURE

The disclosure generally relates to medical devices and, more particularly, relates to medical devices for removing an object from a body.

### BACKGROUND OF THE DISCLOSURE

Snares and forceps are well-known medical devices. Using gastro-enterology as an example, if a sessile polyp (a relatively flat growth on the interior wall of the colon) needs to be removed, an endoscope is first navigated to the point of the procedure to provide the physician with visual access to the procedure. One or more tools are used to grasp and secure the polyp. U.S. Pat. Nos. 5,290,284 and 5,746,747 are two examples of such devices.

The tools used are typically provided as either a forcep or a snare. The forcep typically includes first and second arms which can be manipulated using a guide wire or the like attached to a hand control device. The forcep provides secure grip and retention of the foreign body. However, in order to remove the polyp, the forcep must be pulled away from the colon wall while maintaining a grip on the polyp. When this process is used with a cautery to remove the polyp, the patient can be at increased risk of bleeding and perforation of the colon wall because of a deeper transmural burn caused by the forcep.

Alternatively, a snare, a looped guide wire provided through a catheter, can be used to encircle the polyp. When this process is used with a cautery to remove the polyp, the burn or cautery effect tends to be more superficial, and thus this method may be safer than the forcep. For small sessile polyps, the snare may be used with or without a cautery. This method could provide for the least amount of complications relating to bleeding and perforation because after encircling the polyp, the snare is constricted or pulled so as to sever the polyp away. Such slicing action is less likely to cause hemorrhaging than the forcep, but its grip upon the foreign body is necessarily weak. Thus, the looped guide wire is most often used in conjunction with some sort of gathering basket or screen provided behind the snare to catch the polyp once removed. For example, U.S. Pat. No. 5,997,547 discloses such a basket.

While such instruments are serviceable, the industry continues to call for improvements particularly in the arenas of reliability, safety, and ease of use. For example, with existing combination tools, the snare and forcep are effectively independent devices making navigation to the polypectomy site and securement of the polyp once removed less than optimal. In addition, those devices that do include a forcep or the like for securing the polyp require the physician to actively hold the forcep in a closed position. This necessarily requires the dedicated use of at least one hand, making manipulation of the snare, endoscope and the like a difficult exercise.

### SUMMARY OF THE DISCLOSURE

In accordance with one aspect of the disclosure, an apparatus for removing an object from a body is provided, which may comprise a sheath member, a forcep, a snare, and a biasing member. The sheath member may include a first channel and a second channel. The first and second channels may be disposed in a fixed position, and the first channel may extend further from the sheath member than the second channel. The snare may be slidably disposed in the first channel of the sheath member, and may include a snare loop to encircle the object. The forcep may be slidably disposed in the second channel of the sheath member, and maybe oriented into a threaded position relative to the snare loop prior to engaging the body. The biasing member may be operatively associated with the forcep. That is, the biasing member may be operable to adjust the forcep into first and second positions.

In accordance with another aspect of the disclosure, an apparatus for removing an object from a body is provided, which may comprise a sheath member, a forcep, a snare, a biasing member, and a control member. The sheath member may include a first channel and a second channel. The first and second channels may be disposed in a fixed position, and the first channel may extend further from the sheath member than the second channel. The snare may be slidably disposed in the first channel of the sheath member, and may include a snare loop to encircle the object. The forcep may be slidably disposed in the second channel of the sheath member, and may be oriented into a threaded position relative to the snare loop prior to engaging the body. The biasing member may be operatively associated with the forcep. That is, the biasing member may be operable to adjust the forcep into first and second positions. The control member may include an actuator for overcoming the biasing member, which may be mounted in the control member.

In accordance with another aspect of the disclosure, an apparatus for removing an object from a body is provided, which may comprise a sheath member, a forcep, a snare, means for biasing the forcep, and means for ensuring a threaded position. The sheath member may include a first channel and a second channel. The first and second channels may be disposed in a fixed position, and the first channel may extend further from the sheath member than the second channel. The snare may be slidably disposed in the first channel of the sheath member, and may include a snare loop to encircle the object. The forcep may be slidably disposed in the second channel of the sheath member, and may be oriented into a threaded position relative to the snare loop prior to engaging the body. The biasing member may be operatively associated with the forcep. That is, the biasing member may be operable to adjust the forcep into first and second positions. The means for biasing the forcep may be operable to adjust the forcep into first and second positions. The means for ensuring the forcep is oriented into the threaded position may be operable to adjust into first and second configurations.

In accordance with another aspect of the disclosure, a method for removing an object from a body is provided, which comprises the steps of providing a forcep oriented into a threaded position relative to a snare loop of a snare slidably disposed in a first channel prior to engaging the body, adjusting the forcep between a first position and a second position to grasp the object, adjusting the snare loop via a control member to a first configuration to advance over the forcep grasping the object, and adjusting the snare loop via the control member to a second configuration to secure the object. The forcep is operatively associated with a biasing member operable to engage the forcep in the first and second positions. Further, the forcep is slidably disposed in a second channel. The first and second channels are disposed in a fixed position in a sheath member such that the first channel extends further from the sheath member than the second channel.

These and other aspects and features of the disclosure will become apparent from the following detailed description when taken in conjunction with the accompanying drawings.

### BRIEF DESCRIPTION OF THE DRAWINGS

FIG. 1 is a schematic representation of a medical device constructed in accordance with teachings of the disclosure;

FIG. 2 is a cross-sectional view of the medical device of FIG. 1, taken along line 2-2 of FIG. 1;

FIG. 3 is a schematic representation of a control member constructed in accordance with teachings of the disclosure;

FIG. 4 is a schematic representation of the medical device positioned within a lumen for removal of a sessile polyp, with a forcep depicted in a threaded position through a snare loop;

FIG. 5 is a schematic representation similar to FIG. 4, but depicting the forcep in an open position;

FIG. 6 is a schematic representation similar to FIG. 4, but depicting the forcep in a closed position;

FIG. 7 is a schematic representation similar to FIG. 4, but depicting the snare loop in an open configuration;

FIG. 8 is a schematic representation of the forcep retracting and pulling the sessile polyp through the snare loop;

FIG. 9 is a schematic representation similar to FIG. 8, but depicting the snare loop closing;

FIG. 10 is a schematic representation of the snare loop retracting and after cutting a portion of the sessile polyp;

FIG. 11 is a flow chart depicting a sequence of steps which may be taken according to a method taught by the disclosure.

While the disclosure is susceptible to various modifications and alternative constructions, certain illustrative embodiments thereof have been shown in the drawings and will be described below in detail, it should be understood, however, that there is no intention to limit the disclosure to the specific forms disclosed, but on the contrary, the intention is to cover all modifications, alterative constructions and equivalents falling within the spirit and scope of the disclosure as defined by the appended claims.

### DETAILED DESCRIPTION OF THE PREFERRED EMBODIMENTS

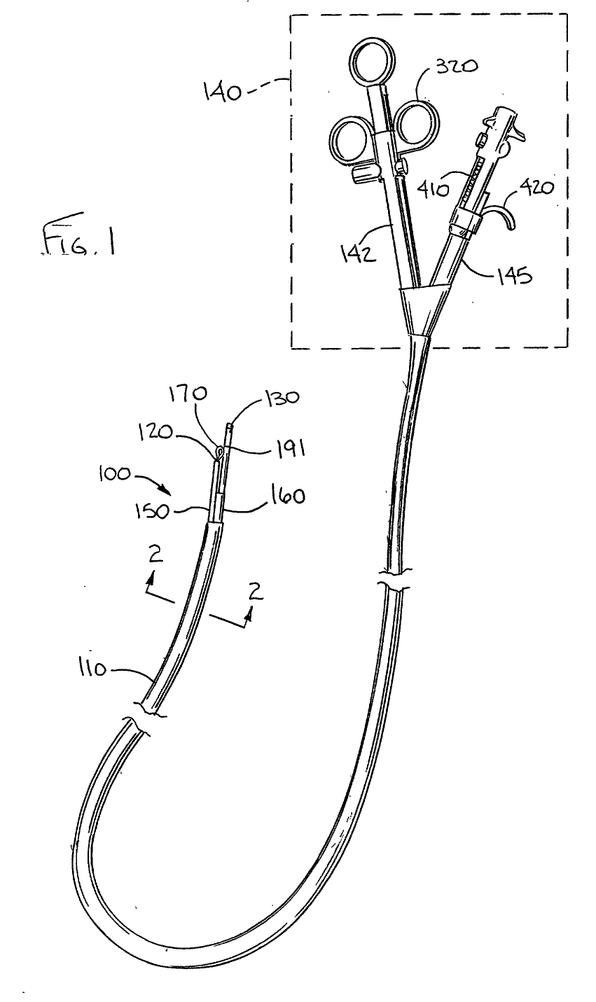

Referring now to the drawings, and with specific reference to FIG. 1, a medical device constructed in accordance with the teachings of the disclosure is generally referred to by reference number 100. While the medical device 100 will be described herein with reference to a device for performing biopsy procedures, particularly removal of sessile polyps from a human colon, it is to be understood that the device can be used for other medical procedures such as, but not limited to, foreign object retrieval from the stomach, esophagus, etc., as well as veterinary procedures of similar type.

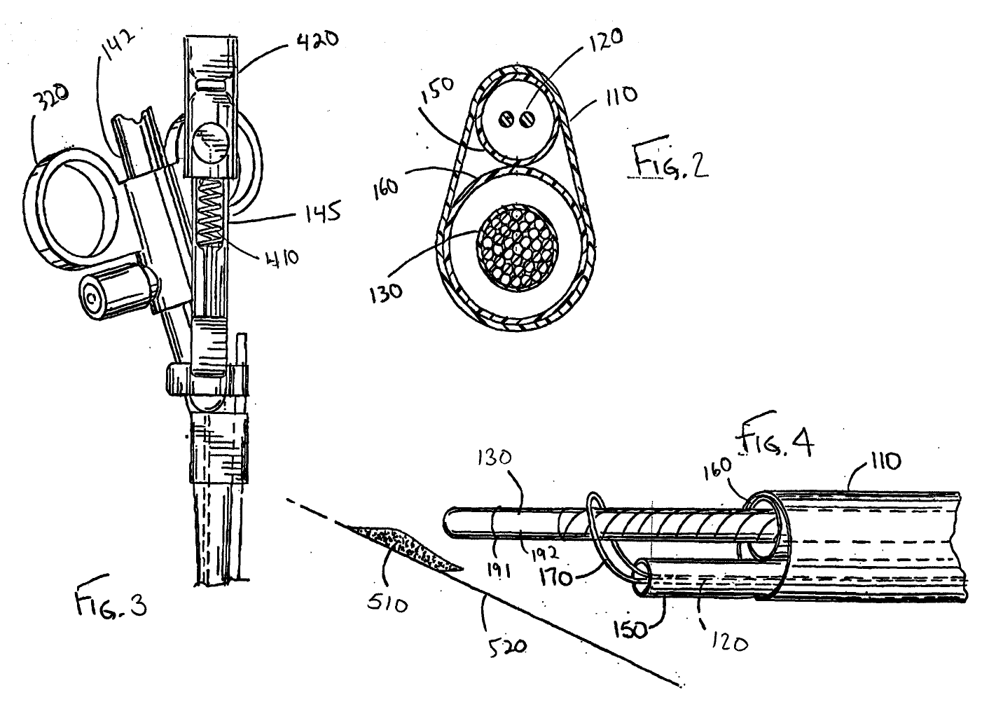

As shown therein, the medical device 100 may generally include a sheath member 110, a snare 120, a forcep 130, and a control member 140. In particular, the sheath member 110 may include a first channel 150 and a second channel 160 as shown in FIG. 2. The sheath member 110, the first channel 150, and the second channel 160 may be constructed from, but are not limited to, a plastic material or an elastic material such as, but not limited to, polyethylene, polypropylene, or the like. The first and second channels 150, 160 are disposed in a fixed position relative to each other and to the sheath member 110. Further, the first channel 150 extends further from the sheath member 110 than the second channel 160 so that the forcep 130 is oriented into a threaded position as described in further detail below.

Referring to FIG. 1 again, the snare 120 is illustrated to further include a snare loop 170. The snare 120 is slidably disposed within the first channel 150, and the forcep 130 is slidably disposed within the second channel 160. As noted above, the first channel 150 and the second channel 160 are in fixed positions with the first channel 150 extending further from the sheath member 110 than the second channel 160 so that the forcep 130 is oriented into a threaded position relative to the snare loop 170 prior to engaging the body, defined herein as a position wherein the snare loop 170 circumnavigates the forcep 130. For example, the first channel 150 may extend 0.375 inches further from the sheath member 110 than the second channel 160, and the forcep 130 may be disposed 0.3125 inches beyond the first channel 150 from the second channel 160. Thus, the forcep 130 is disposed through the snare loop 170 of the snare 120, i.e., in the threaded position.

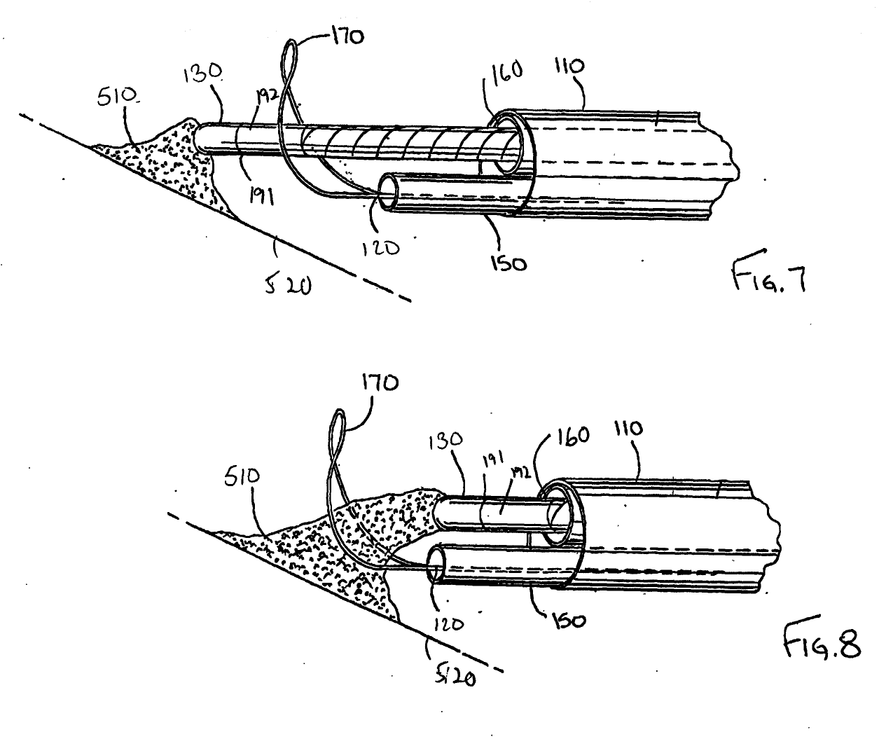

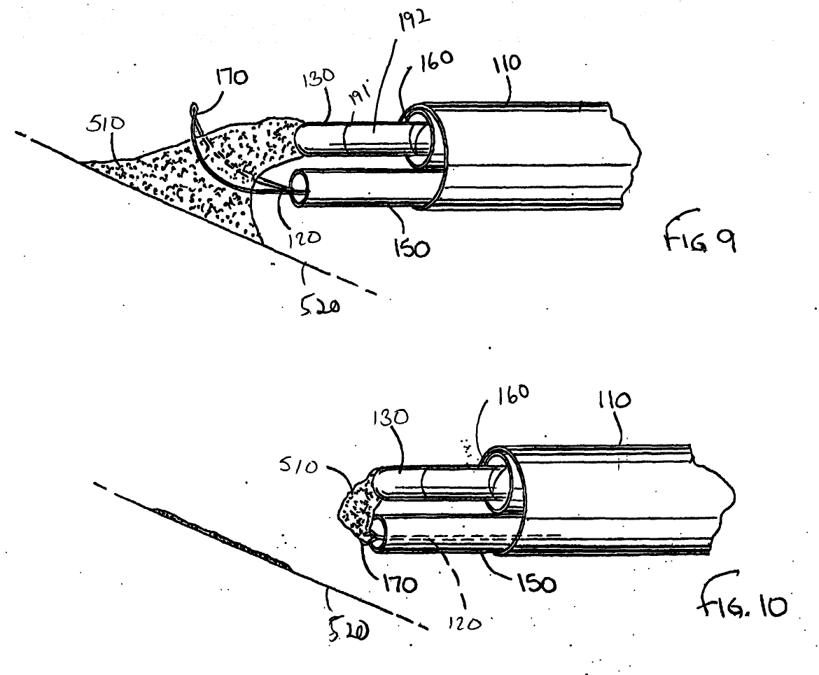

The snare 120 and the forcep 130 may be controlled by the control member 140, which itself may include first and second control members 142, 145, respectively. The first control member 142 may be, but is not limited to, a syringe-like control member operatively coupled to the snare 120 to adjust the snare loop 170 to a first configuration (e.g., an open configuration) as depicted-in FIG. 7 and a second configuration (e.g., a closed configuration) as depicted in FIG. 9. For example, as shown in FIG. 3, the snare loop 170 may be adjusted to a closed configuration in response to depression of an actuator such as a trigger 320 of the first control member 142, i.e., squeezing a plunger of the syringe-like control member 142. The snare loop 170 operates as a lasso or a noose to advance over and secure an object as shown best in FIGS. 8 and 9. The snare loop 170 may be a wire of suitable metal or plastic material having memory characteristics. More specifically, the snare loop 170 may be selectively deformed so as to have a natural, unstressed state allowing for expansion as shown in FIG. 8, and may be held in a more linear configuration under the constricting stress imparted by the channel 150. In so doing, when in open configuration, the snare loop 170 expands its circumference and bends towards the sheath member 110 so that an object, e.g., polyp, may be lassoed by the snare loop 170. In the closed configuration, the snare loop 170 reduces its size to form a tight fit around an object grasped by the forcep 130.

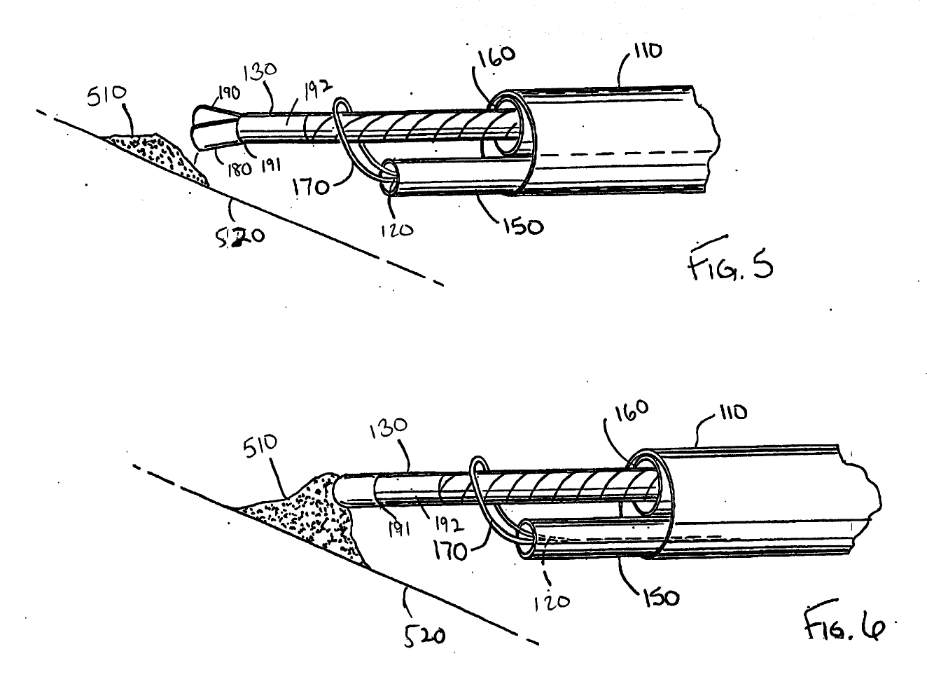

The forcep 130 may generally include a first arm 180 and a second arm 190. The second control member 145, which is further described in detail below, is operatively coupled to the forcep 130 to control the first and second arms 180, 190 of the forcep 130. That is, the second control member 145 adjusts the forcep 130 to a first position (e.g., a closed position) as depicted in FIG. 4 and a second position (e.g., an open position) as depicted in FIG. 5. For example, the first and second arms 180, 190 maybe hinged to a sliding pivot 191 and a housing 192 with a control wire (not shown) being connected to the sliding pivot 191. Retraction of the wire may pull the pivot 191 in and close the arms while extension of the pivot 191 may cause the arms 180, 190 to open.

Referring to FIG. 3, an embodiment of the second control member 145 is shown in more detail to include a biasing member 410. The second control member 145 maybe, but is not limited to, a hand-held control member. The biasing member 410 may be, but is not limited to, a spring. The biasing member 410 is operatively coupled to the forcep 130 to adjust the first and second arms 180, 190 of the forcep 130 to a closed position and an open position. In the closed position, the first arm 180 and the second arm 190 and disposed together. In response to depression of a trigger 420 of the second control member 145, the biasing member 410 may adjust the forcep 130 to the open position such that the first and second arms 180, 190 are separated relative to each other. For example, the depression of the trigger 420 may cause the control wire to extend through the channel 160, thereby sliding the pivot 191 (shown in FIG. 4) and opening the arms 180, 190. Accordingly, the forcep 130 is operable to engage an object. By releasing the second control member 145, the biasing member 410 expands and thereby adjusts the forcep 130 to the closed position by retracting the control wire and pulling the pivot 191 so as to close the arms 180, 190. The first and second arms 180, 190 are able to engage the object, i.e., the object is disposed between the first and second arms 180, 190. As a result, the forcep is automatically biased in a closed, gripping position thereby allowing the user of the medical device 100 to have a free hand to perform other tasks without having to bold the second control member 145 to keep a grasp of the object with the forcep 130.

It is to be understood that the above materials and dimensions are provided for the purpose of disclosing the currently known best mode for practicing the teachings of the disclosure, and should not be construed as limiting in any manner.

As noted above the medical device 100 may be operable to secure and remove an object from a body such as, but not limited to, a tumor and a sessile polyp from human tissue such as a large intestine or the like. For illustration purposes, a basic flow for securing and removing a portion of sessile polyp is illustrated pictorially in FIGS. 4-10, and in flowchart format in FIG. 11. Referring first to FIG. 4, the medical device 100 may be operable to secure and remove a portion of a sessile polyp 510 from a wall 520 of an organ, lumen, intestine, etc. As noted above, the forcep 130 is oriented into the threaded position prior to engaging the body such that the forcep 130 extends through the snare loop 170 of the snare 120, i.e., the forcep 130 is threaded through the snare loop 170 as a string is threaded through a needle. Further, to facilitate navigation of the device 100 to the location of the polyp 510, the biasing member 410 of the second control member 145 biases the forcep 130 into a closed position, i.e., the first and second arms 180, 190 are disposed together. The medical device 100 can then be more easily advanced to the sessile polyp 510. Upon arriving at the polyp 510, the forcep 130 is operable to grasp the sessile polyp 510. More specifically, in response to depression of the trigger 420 of the second control member 145, the biasing member 410 adjusts the forcep 130 to an open position as shown in FIG. 5. That is, the first and second arms 180, 190 of the forcep 130 are separated relative to each other when the forcep 130 is adjusted to the open position. Referring to FIG. 6, the medical device 100 can then be further advanced with the second control member 145 then being released so that the biasing member 410 adjusts the forcep 130 back to the closed position to grasp a portion of the sessile polyp 510. The biasing member 410 allows a user to release the trigger 420 of the second control member 145 (i.e., free up a hand to perform other tasks) and still maintain a secure grasp on the portion of the sessile polyp 510 being removed.

After securing the polyp 510 in the forcep 130, and retracting the device slightly to pull the polyp away from the wall 520 as shown in FIG. 6, the snare 120 can be extended by the first control member 142 to adjust the snare loop 170 to the open configuration for the forcep 130 as shown in FIG. 7. When the snare loop 170 is in the open configuration, it bends towards the sheath member 110 to advance over the forcep 130 grasping the sessile polyp 510 in a lasso-like maneuver.

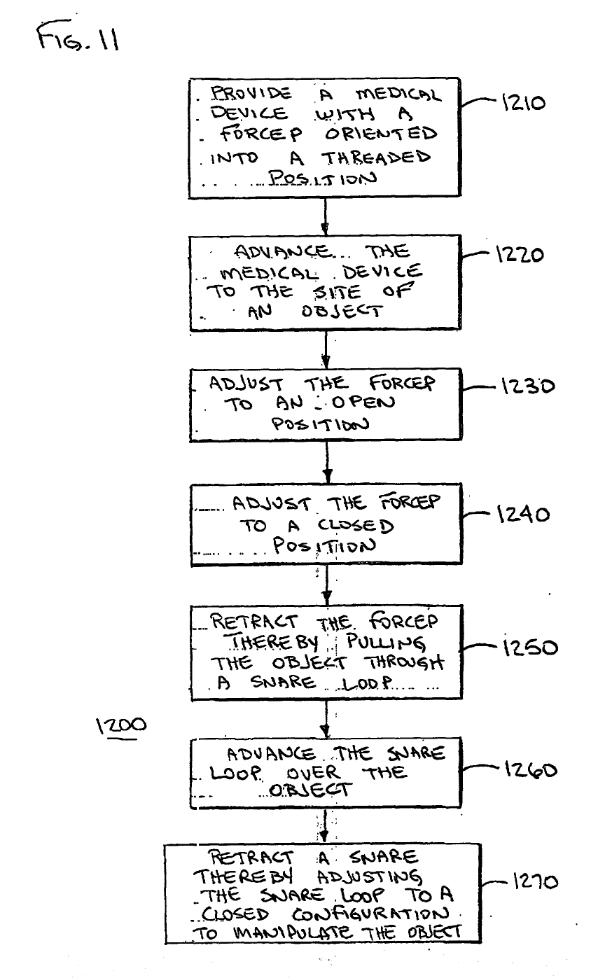

Referring to FIG. 8, the second control member 145 can then be retracted such that the forcep 130 retracts into the second channel 160, thereby pulling the polyp 510 through the snare loop 170. The snare loop 170 may then be retracted as shown in FIG. 9, so as to cut through the polyp 510 and remove a portion of the sessile polyp 510 from the wall 520 of the body. As a result, the forcep 130 is no longer in the threaded position relative to the snare loop 170. The first control member 142 retracts the snare 120 so that the snare loop 170 is in the closed configuration (i.e., the snare loop 170 tightens around the portion of the sessile polyp 510) to remove the portion of the sessile polyp 510 from the wall of the organ 520 as shown in FIGS. 9 and 10. After cutting, the first and second arms 180, 190 of the forcep 130 maintain a grasp on the portion of the sessile polyp 510 as the medical device 100 is retracted from the body. In so doing, the snare is defined herein as manipulating the polyp 510. Manipulating may also take the form of, for example, grasping a foreign object for removal from the body.

Referring now to FIG. 11, the flow chart therein depicts a sequence of steps 1200 which may be taken in accordance with the teachings of the disclosure to remove an object from a body. As shown therein, a first step 1210 thereof may be to provide a medical device with a forcep oriented into a threaded position relative to a snare loop of a snare slidably disposed in a first channel. The forcep is slidably disposed in a second channel. The first and second channels are disposed in a fixed position in a sheath member with the first channel extending further from the sheath member than the second channel so that the forcep is oriented into the threaded position relative to the snare loop. At step 1220, the medical device is advanced to the site of the object through an endoscope or similar device. At step 1230, the forcep is adjusted to an open position by a biasing member. That is, first and second arms of the forcep are separated relative to each other when the forcep is adjusted to the open position. At step 1240, the forcep is further advanced to the object with a control member then being released so that the biasing member adjusts the forcep back to the closed position to grasp the object. The biasing member allows a user to release a trigger of the control member (i.e., free up a hand to perform other tasks) and still maintain a secure grasp on the object being removed.

After securing the object in the forcep, the forcep is retracted into a second channel thereby pulling the object through the snare loop at step 1250. As a result, the forcep is no longer in the threaded position relative to the snare loop. At step 1260, the snare is extended to adjust the snare loop to the open configuration for performing a lasso-like maneuver to the object and the forcep. When the snare loop is in the open configuration, it bends towards the sheath member to advance over the forcep grasping the object. At step 1270, the snare is retracted so that the snare loop is in the closed configuration (i.e., the snare loop tightens around the object) to manipulate the object. That is, the manipulation of the object by the snare loop may be, but is not limited to, severing the object and securing the object.

From the foregoing, one of ordinary skill in the art will appreciate that the disclosure provides a method for removing an object from a body and an apparatus for accomplishing same.

What is claimed is:

1. An apparatus for removing an object from a body, the apparatus comprising:
a sheath member having first and second channels, the first and second channels being disposed in a fixed position, the first channel extending further from the sheath member than the second channel;
a snare slidably disposed in the first channel of the sheath member, the snare having a snare loop;
a forcep slidably disposed in the second channel of the sheath member, the forcep oriented into a threaded position relative to the snare loop prior to engaging the body; and
a biasing member operatively associated with the forcep, the biasing member operable to adjust the forcep into first and second positions.

2. The apparatus of claim 1, wherein the sheath member comprises one of a plastic sheath member and an elastic sheath member.

3. The apparatus of claim 1, wherein the forcep comprises first and second spring-biased arms.

4. The apparatus of claim 1, wherein the snare comprises a wire snare.

5. The apparatus of claim 1, wherein the first position comprises an open position.

6. The apparatus of claim 1, wherein the second position comprises a closed position.

7. The apparatus of claim 1, wherein the biasing member comprises a spring.

8. The apparatus of claim 1, further including first and second control members, the first control member operatively associated with the snare, and the second control member operatively associated with the forcep.

9. The apparatus of claim 1, wherein the object comprises living tissue.

10. The apparatus of claim 9, wherein the living tissue is one of a tumor and a sessile polyp.

11. An apparatus for removing an object from a body, the apparatus comprising:
a sheath member having first and second channels, the first and second channels being disposed in a fixed position;
a snare slidably disposed in the first channel of the sheath member, the snare having a snare loop;
a forcep slidably disposed in the second channel of the sheath member, the first channel extending further from the sheath member than the second channel so that the forcep is oriented into a threaded position relative to the snare loop prior to engaging the body;
a biasing member operatively associated with the forcep, the biasing member operable to adjust the forcep into first and second positions; and
a control member having an actuator for overcoming the biasing member, the biasing member being mounted in the control member.

12. The apparatus of claim 11, wherein the sheath member comprises one of a plastic sheath member and an elastic sheath member.

13. The apparatus of claim 11, wherein the forcep comprises first and second spring-biased arms.

14. The apparatus of claim 11, wherein the snare comprises a wire snare.

15. The apparatus of claim 11, wherein the first position comprises an open position.

16. The apparatus of claim 11, wherein the second position comprises a closed position.

17. The apparatus of claim 11, wherein the biasing member comprises a spring.

18. The apparatus of claim 11, wherein the control member further includes a control member operatively associated with the snare.

19. The apparatus of claim 11, wherein the object comprises living tissue.

20. The apparatus of claim 19, wherein the living tissue is one of a tumor and a sessile polyp.

21. An apparatus for removing an object from a body, the apparatus comprising:
a sheath member having first and second channels, the first and second channels being disposed in a fixed position;
a snare slidably disposed in the first channel of the sheath member, the snare having a snare loop;
a forcep slidably disposed in the second channel of the sheath member, the first channel extending further from the sheath member than the second channel so that the forcep oriented into a threaded position relative to the snare loop prior to engaging the body;
means for biasing the forcep into first and second positions; and
means for ensuring the forcep is oriented into the threaded position.

22. The apparatus of claim 21, wherein the sheath member comprises one of a plastic sheath member and an elastic sheath member.

23. The apparatus of claim 21, wherein the snare comprises a wire snare.

24. The apparatus of claim 21, wherein the means for biasing the forcep comprises a spring.

25. The apparatus of claim 21, wherein the object comprises living tissue.

26. The apparatus of claim 25, wherein the living tissue is one of a tumor and a sessile polyp.

27. A method for removing an object from a body, the method comprising:
providing a forcep oriented into a threaded position relative to a loop of a snare slidably disposed in a first channel, the forcep being slidably disposed in a second channel, the first and second channels being disposed in a fixed position in a sheath member, and the first channel extending further from the sheath member relative to the second channel so that forcep is oriented into the threaded position relative to the loop prior to engaging the body;
adjusting the forcep between a first position and a second position to grasp the object, the forcep being operatively associated with a biasing member;
adjusting the snare loop via a control member to a first configuration to advance over the forcep and encircle the object; and
adjusting the snare loop via the control member to a second configuration to manipulate the object.

28. The method of claim 27, wherein the forcep is provided within living tissue to remove one of a tumor and a sessile polyp.

29. The method of claim 27, wherein adjusting the snare loop via the control member to the first configuration causes the snare loop to expand and bend toward the sheath member into an open configuration.

30. The method of claim 27, wherein adjusting the snare loop via the control member to the second configuration causes the snare loop to constrict into a closed configuration.

31. The method of claim 27, wherein adjusting the snare loop via the control member to a second configuration causes the snare loop to one of sever the object and secure the object.
